(19) United States
(12) Patent Application Publication
(10) Pub. No.: US 2008/0154141 A1
(43) Pub. Date: Jun. 26, 2008

(54) Intravascular Flow Sensor

(75) Inventors: Allan Charles Shuros, St. Paul, MN (US); Michael J. Kane, Lake Elmo, MN (US)

Correspondence Address: PAULY, DEVRIES SMITH & DEFFNER, L.L.C., PLAZA VII- SUITE 3000, 45 SOUTH SEVENTH STREET, MINNEAPOLIS, MN, 55402-1630, US

(73) Assignee: CARDIAC PACEMAKERS, INC., St. Paul, MN (US)

(21) Appl. No.: 11/616,073

(22) Filed: Dec. 26, 2006

(51) Int. Cl. A61B 5/026

(52) U.S. Cl. 600/505

(57) ABSTRACT

Disclosed herein, among other things, are intravascular flow sensors and related methods. In an embodiment, the invention includes an intravascular flow sensor including a strain gauge and a positioning element configured to be expandable from a first diameter to a second diameter. In an embodiment, the invention includes an intravascular flow sensor including a deflection member configured to be positioned within a lumen defined by a tissue wall, the deflection member including a flexible shaft and a shaft tip; and a positioning member configured to prevent the shaft tip from contacting the tissue wall. In an embodiment, the invention includes an implantable medical device including a pulse generator and an intravascular flow sensor in communication with the pulse generator, the intravascular flow sensor including a strain gauge. Other aspects and embodiments are provided herein.

### TECHNICAL FIELD

This disclosure relates generally to flow sensors and, more particularly, to intravascular flow sensors and related methods.

### BACKGROUND OF THE INVENTION

Measuring the velocity of blood flow through veins and arteries of the body can be an important step in the diagnosis and monitoring of various health problems. By way of example, a sudden decrease in the velocity of blood flow occurring during all phases of the cardiac pumping cycle can be indicative of a significant occlusion requiring immediate medical intervention. Blood flow velocity data can also be useful when monitoring progressive diseases such as heart failure. For example, a long term decline in peak blood flow velocities may correlate with a negative prognosis for a heart failure patient. In addition, because blood flow velocities change during different phases of the cardiac pumping cycle, blood flow velocity information can also be useful for monitoring cardiac rhythm problems.

Accordingly, there is a need for systems and methods of measuring intravascular fluid flow.

### SUMMARY OF THE INVENTION

This disclosure relates generally to flow sensors and, more particularly, to intravascular flow sensors and related methods. In an embodiment, the invention includes an intravascular flow sensor including a strain gauge; and a positioning element operably connected to the strain gauge, the positioning element configured to be expandable from a first diameter to a second diameter, the second diameter larger than the first diameter.

In an embodiment, the invention includes an intravascular flow sensor including a deflection member configured to be positioned within a lumen defined by a tissue wall, the deflection member including a flexible shaft and a shaft tip, the deflection member configured to generate a signal corresponding to flexion of the flexible shaft; and a positioning member configured to prevent the shaft tip from contacting the tissue wall.

In an embodiment, the invention includes an implantable medical device including a pulse generator and an intravascular flow sensor in communication with the pulse generator, the intravascular flow sensor including a strain gauge.

In an embodiment, the invention includes an implantable medical device including a deformable enclosed volume and a sensor disposed within the enclosed volume, wherein deformation of the enclosed volume results in the generation of a signal.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope of the present invention is defined by the appended claims and their legal equivalents.

### BRIEF DESCRIPTION OF THE DRAWINGS

The invention may be more completely understood in connection with the following drawings, in which:

FIG. 1 is schematic side view of a flow sensor disposed within a vascular lumen in accordance with an embodiment of the invention.

FIG. 2 is a schematic front view of a flow sensor taken along line 2-2′ of FIG. 1.

FIG. 3 is a schematic side view of a flow sensor in accordance with an embodiment of the invention.

FIG. 4 is a schematic side view of a flow sensor in accordance with another embodiment of the invention.

FIG. 5 is a schematic side view of a flow sensor in accordance with another embodiment of the invention.

FIG. 6 is a schematic diagram of a flow sensor in conjunction with a CRM device in accordance with an embodiment of the invention.

FIG. 7 is a schematic diagram of a flow sensor in conjunction with a CRM device in accordance with another embodiment of the invention.

FIG. 8 is a schematic side view of a flow sensor in accordance with another embodiment of the invention.

FIG. 9 is a schematic front view of a flow sensor taken along line 9-9′ of FIG. 8.

FIG. 10 is a schematic side view of a flow sensor in accordance with another embodiment of the invention.

FIG. 11 is a schematic side view of a flow sensor in accordance with another embodiment of the invention.

FIG. 12 is a schematic side view of the flow sensor of FIG. 11 with the positioning element in an unexpanded configuration.

FIG. 13 is a schematic side view of a flow sensor in accordance with another embodiment of the invention.

FIG. 14 is a schematic side view of a flow sensor in accordance with another embodiment of the invention.

FIG. 15 is a schematic side view of a flow sensor in accordance with another embodiment of the invention.

FIG. 16 is a schematic side view of a flow sensor in accordance with another embodiment of the invention.

FIG. 17 is a schematic side view of a flow sensor in accordance with another embodiment of the invention.

FIG. 18 is a schematic side view of a flow sensor in accordance with another embodiment of the invention.

FIG. 19 is a schematic side view of a flow sensor in accordance with another embodiment of the invention.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

Blood flow velocity can be an important piece of information to gather when diagnosing and/or monitoring various cardiovascular health problems. The use of intravascular blood flow sensors can be a particularly desirable way to gather blood flow velocity data. Specifically, intravascular flow sensors can be advantageous because they can be implanted within a patient so that monitoring of the blood flow velocity can be done continuously, semi-continuously, or on-demand and the data can be tracked over a period of time. This is in contrast to flow sensors, such as some ultrasound based flow sensors, that can only be used within a care facility such as within a hospital or a cardiac catheterization lab. In addition, intravascular flow sensors can be advantageous because well-established minimally invasive percutaneous surgical techniques can be used to implant them. Embodiments of the present invention include intravascular flow sensors and methods of making and using the same.

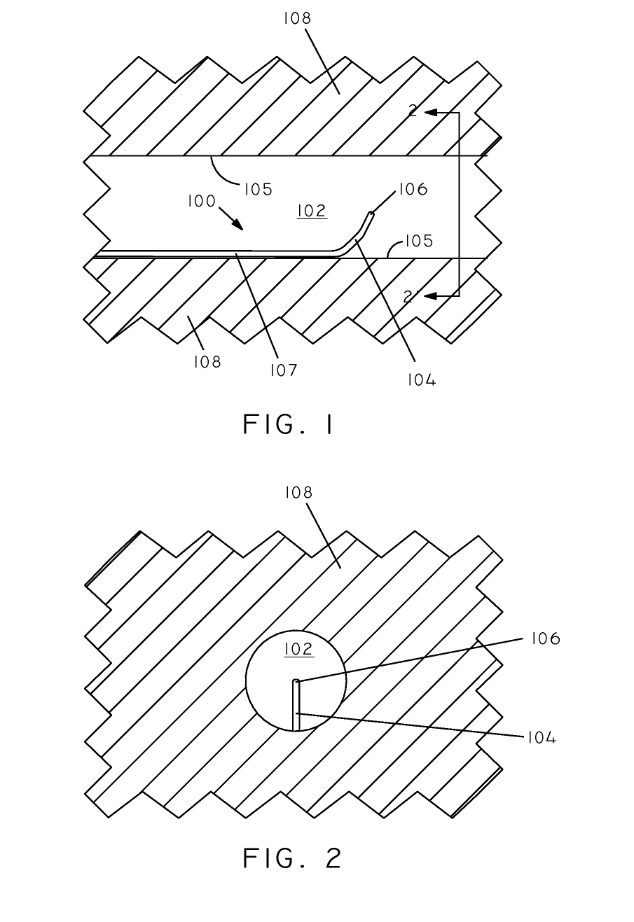

Referring now to FIG. 1, an intravascular flow sensor 100 in accordance with an embodiment of the invention is shown disposed within an exemplary vascular lumen 102. The vascular lumen 102 passes through bodily tissue 108 and is defined by a lumen wall 105 that is continuous around the lumen 102. The intravascular flow sensor 100 includes a connecting shaft 107, a flexible shaft 104, and a tip 106. Together, the flexible shaft 104 and the tip 106 can be referred to as a deflection member. The flexible shaft 104 can be curved. The connecting shaft 107 and the flexible shaft 104 can take on various shapes in cross-section including circular, oval, square, a rectangular, polygonal, or irregular. The tip 106 of the sensor 100 can be positioned within the central area of the vascular lumen 102. Passing blood flow creates drag on the flexible shaft 104 and on the tip 106. This drag force can be sufficient to cause the flexible shaft 104 to bend. The degree to which the flexible shaft bends (or the flexion of the flexible shaft 104) can be captured as an optical and/or electric signal. This signal can then be evaluated in order to estimate the blood flow velocity within the vascular lumen 102.

The connecting shaft 107 and the flexible shaft 104, can include various materials such as metals, polymers, or the like. The connecting shaft 107 and the flexible shaft 104 can be made of materials that are biocompatible and/or non-thrombogenic. In some embodiments, the flexible shaft 104 is more flexible than the connecting shaft 107.

FIG. 2 is a frontal view of an intravascular flow sensor taken along line 2-2′ of FIG. 1. In this view, the tip 106 is shown disposed near the center of the vascular lumen 102. While not intending to be bound by theory, the flow velocity as measured can be affected by the position within the lumen 102 where the velocity measurement is taken. Based on the effects of drag exerted by the tissue walls 105, the highest flow velocities would be expected in the middle of the vascular lumen 102 while the lowest flow velocities would be expected near the walls 105 of the vascular lumen 102. In some embodiments, the intravascular flow sensor is configured so that the tip 106 is disposed in or around the center of the vascular lumen 102.

The intravascular flow sensor can be disposed within any vascular lumen in the body as desired, including both arteries and veins. By way of example, the intravascular flow sensor can be disposed within the pulmonary artery, the renal artery, the renal vein, the coronary sinus, etc. While not intending to be bound by theory, the pulmonary artery can be a desirable place to locate the intravascular flow sensor at least because pulmonary artery blood flow can be correlated with blood flow through the left ventricle of the heart which can be important information for diagnosis and treatment of various conditions.

The specific diameter of the vascular lumen 102 will depend on various factors including the phase of the cardiac cycle (systolic diameter vs. diastolic diameter), the location of the vascular lumen 102 within the body, the disease status of the particular patient, etc. The largest artery in the body is the aorta which has a diameter of up to about 2.5 cm in healthy patients and can get as large as 4.0 cm in diseased patients. In an embodiment, the intravascular flow sensor 100 of the invention is disposed within a vascular lumen having a diameter of less than about 5.0 centimeters in diameter. The intravascular flow sensor 100 of the invention can also be disposed in other areas of the body. By way of example, the intravascular flow sensor 100 of the invention can be disposed directly within one of the four chambers of the heart.

The degree of flexion of the flexible shaft 104 is related to the degree of drag force exerted on the flexible shaft 104 and the tip 106 by passing fluid flow. The drag force (F_{D}) exerted is related to the velocity of the fluid flow according to the drag equation:

$${F}_{D} = \frac{{C}_{d} A ρ {V}^{2}}{2}$$

wherein C_{d}=the drag coefficient; A=the effective area of the surface in the flow of fluid (such as the cross-sectional area of the flexible shaft and tip); ρ=the fluid density; and V=the fluid velocity at the point of measurement. As such, one way of estimating the velocity of fluid flow is to relate the degree of flexion of the flexible shaft 104 to the fluid velocity using the drag equation. For purposes of calibration, the intravascular flow sensor 100 can be placed within a vascular lumen in conjunction with another flow sensor (calibration sensor) that is known to be reasonably accurate. The calibration flow sensor can be intravascular or extravascular. The degree of flexion of the flexible shaft 104 can be recorded in conjunction with the flow measurements from the calibration sensor at both diastole (relatively low flow velocity, particularly in most arteries) and systole (relatively high flow velocity, particularly in most arteries). In some embodiments, the calibration flow sensor can include equipment for performing pulsed or continuous wave Doppler ultrasound. After the intravascular flow sensor 100 is calibrated, a processor can be used to convert flexion (bending) data to blood flow velocity data.

In some embodiments, such as where the blood flow sensor is implanted in a vascular lumen subject to highly pulsatile blood flow, a re-calibration procedure can be performed periodically when the blood flow velocity through the particular vascular lumen is known to be very low or zero, such as during diastole. For example, the degree of flexion exhibited by the intravascular flow sensor during diastole can be taken to indicate zero flow velocity. Recalibration of the flow sensor in this manner can prevent chronic drift of the blood flow velocity data over time.

In will be appreciated that flexion of the flexible shaft can be detected in various ways including both optically and electrically. In some embodiments, the flow sensor includes an electrical strain gauge. In other embodiments, the flow sensor includes an optical strain gauge. Specifically, in some embodiments the flexible shaft can include an optical conductor such as an optical fiber. Optical fibers generally include a core surrounded by a cladding layer. To confine the optical signal to the core, the refractive index of the core is typically greater than that of the cladding. The boundary between the core and cladding may either be abrupt, in step-index fiber, or gradual, in graded-index fiber. Optical signals can pass through the core of the optical fiber by means of total internal reflection. However, if the angle of incidence of light striking the boundary between the core and cladding exceeds a critical value, then some amount of the optical signal will pass outside of the optical fiber and not be reflected internally. As such, an optical fiber that is bent beyond a critical angle will exhibit some degree of optical signal loss. Therefore, bending of an optical fiber can be detected by monitoring the optical signals transmitted by the optical fiber.

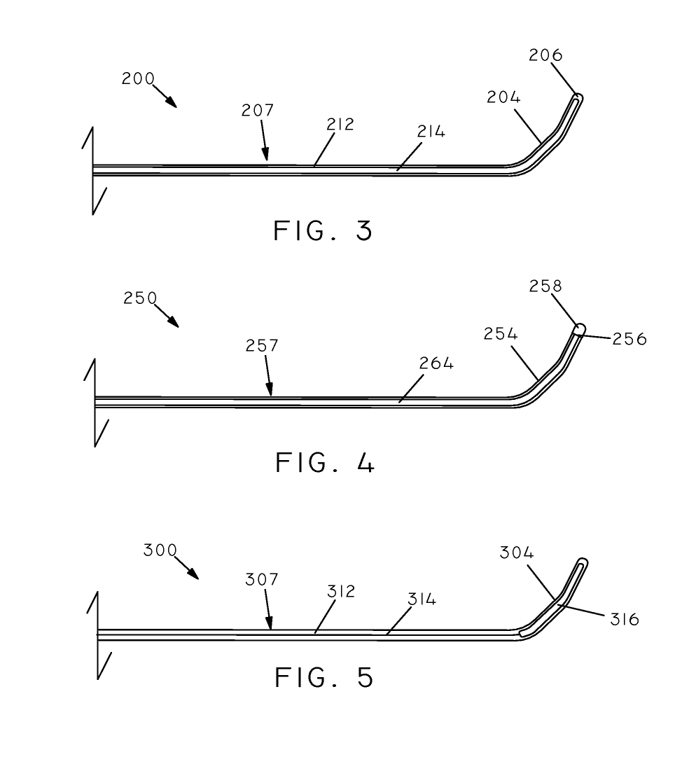

FIG. 3 shows a schematic side view of an intravascular flow sensor 200 in accordance with an embodiment of the invention. The flow sensor 200 includes a connecting shaft 207. The connecting shaft 207 includes a sheath 212 and an optical conductor 214. The optical conductor 214 can also pass through the flexible shaft 204. An optical signal, such as that generated by a light emitting diode (LED) is passed through the optical conductor 214, reflected at the tip 206, passed back through the optical conductor 214 and then received by a component (not shown) such as a photodiode. As described above, flexion (bending) of the flexible shaft 204 can affect the optical absorbance of light passing through the optical conductor 214. As such, flexion of the flexible shaft 204 can be detected by monitoring the intensity of the optical signal passing through the optical conductor 214.

In some embodiments, the optical conductor 214 includes an unmodified optical fiber. However, in some embodiments, portions of the optical fiber cladding can be removed to enhance sensitivity of specific regions of the optical fiber to bending signal loss. For example in some embodiments, an optical fiber with a cladding passes through the connecting shaft 207 and the flexible shaft 204 of the flow sensor. However, a portion of the cladding on the fiber is removed or otherwise modified where the optical fiber passes through the flexible shaft 204.

In some embodiments, the optical conductor 214 includes a bend-enhanced fiber (BEF) sensor. BEF sensors are curvature-measuring optical analogs of elongation-measuring resistance strain gauges. BEFs can be made by treating optical fibers to have an optically absorptive zone along a thin axial stripe. Light transmission through the fiber past this zone then becomes a robust function of curvature that is more sensitive to bending than otherwise similar untreated optical fiber.

The sheath 212 can include various materials such as metals, polymers, coatings, pigments, etc. In some embodiments, the sheath 212 can be configured to physically isolate the optical conductor 214 from the fluids in the vascular lumen. In many embodiments, the sheath 212 is disposed against a wall of a vascular lumen. The sheath 212 can be impregnated or coated with various materials, such as drug eluting materials, to achieve various purposes such as increased lubricity, decreased thrombogenicity, enhanced biocompatibility, decreased scaring or tissue growth, increased tissue growth, prevention of stenosis, etc. In some embodiments, the sheath 212 can include a layer of polytetrafluoroethylene.

The optical conductor 214 can be used to convey optical signals bidirectionally. For example, optical signals can be passed through the optical conductor 214 towards the tip 206. At the same time, optical signals can be passed back from the tip 206 through the optical conductor 214 and then on for further processing.

In some cases, a secondary sensing element can be affixed to the flow sensor to provide additional data. For example, a secondary sensing element, such as an optical chemical sensor, can be affixed to the tip and provide information regarding various aspects of the current physiological state of an organism. In such an embodiment, concentrations of analytes can be measured using the secondary sensing element. Analytes sensed by the secondary sensing element can include ions (cation or anion) and non-ions. Specific examples of analytes that can be sensed include acetic acid (acetate), aconitic acid (aconitate), ammonium, amphetamine, blood urea nitrogen (BUN), B-type natriuretic peptide (BNP), bromate, bupivacaine, calcium, carbon dioxide, cardiac specific troponin, chloride, choline, citric acid (citrate), cortisol, copper, creatinine, creatinine kinase, ephedrine, ethanol, fluoride, formic acid (formate), glucose, hydronium ion (pH), isocitrate, lactic acid (lactate), lidocaine, lignocaine, lithium, magnesium, maleic acid (maleate), malonic acid (malonate), myoglobin, nitrate, nitric-oxide, norephedrine, ethanol, oxalic acid (oxalate), oxygen, phosphate, phthalate, potassium, prilocaine, procaine, protamine, pyruvic acid (pyruvate), salicylate, selenite, sodium, sulfate, urea, uric acid, and zinc. In a specific embodiment, analytes that can be sensed with the secondary sensing element include potassium, sodium, chloride, calcium, and hydronium (pH). Hematocrit refers to the proportion of blood volume that is occupied by red blood cells. In some embodiments, hematocrit can be measured by the secondary sensing element.

FIG. 4 shows a schematic side view of a flow sensor 250 with a secondary sensing element 258 affixed to the tip 256. The secondary sensing element 258 is in optical communication with an optical conductor 264 passing through the connecting shaft 257 of the flow sensor 250. Thus, optical signals for interrogation of the secondary sensing element 258 as well as optical signals representing the output from the secondary sensing element 258 can both be passed through the optical conductor. The signals produced by the secondary sensing element 258 can be in addition to the signals related to flexion of the flexible shaft 254.

While not intending to be bound by theory, it is believed that there are advantages associated with a flow sensor that detects flow using an optical approach instead of an electric approach. Generally, the materials used to construct an optically based flow sensor are more biocompatible than some materials (such as electrically conductive materials) used to construct an electric flow sensor. Biocompatibility is particularly important in the context of an implanted intravascular flow sensor. In addition, optically based flow sensors can generally be less susceptible to interference associated with MRI (magnetic resonance imaging) testing. By way of example, the time-varying magnetic field gradients produced by MRI test equipment can induce currents in electrically conductive materials. However, optically based flow sensors can be constructed with a minimum of electrically conductive materials and thus are generally not subject to MRI induced currents to the same degree as electrically based flow sensors.

However, in some embodiments, the flexible shaft of the flow sensor can generate electrical signals that correspond to the velocity of fluid flow. As used herein, electrical signals can include changes in electrical properties such as voltage, current, resistance, and the like. In some embodiments, the flexible shaft of the flow sensor includes an electrical strain gauge. FIG. 5 shows a schematic side view of an intravascular flow sensor 300 in accordance with an embodiment of the invention. The flow sensor 300 includes a connecting shaft 307 and a flexible shaft 304. The connecting shaft 307 includes a sheath 312 and an electrical conductor 314. The flexible shaft 304 includes an electrical strain gauge 316 in electrical communication with the electrical conductor 314. The electrical strain gauge 316 can be configured to generate an electrical signal corresponding to the degree of flexion of the flexible shaft 304. By way of example, the electrical strain gauge 316 can include a piezoresistor that changes its resistance when bent. In some embodiments, the electrical strain gauge 316 can include a metallic foil type strain gauge. In some embodiments, the electrical strain gauge 316 can include a silicon type strain gauge. The signal generated by the electrical strain gauge 316 then passes through, or is sensed through, electrical conductor 314 and onto a processor (not shown) for further analysis.

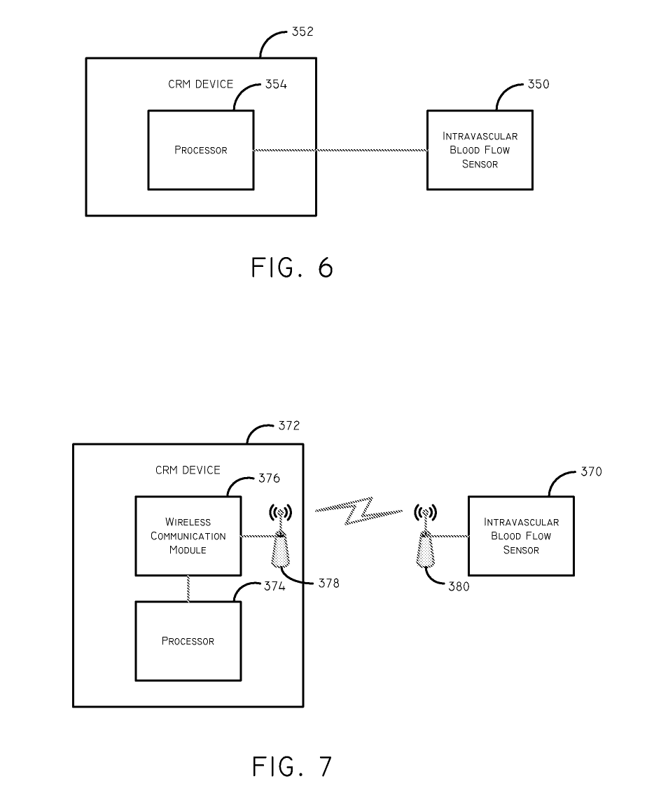

FIG. 6 shows a schematic diagram of an intravascular flow sensor 350 in conjunction with a cardiac rhythm management (CRM) device 352. CRM devices can include pacemakers, cardiac resynchronization therapy (CRT) devices, remodeling control therapy (RCT) devices, cardioverter/defibrillators, pacemaker-cardioverter/defibrillator, and the like. One example of a CRM device is described in commonly assigned U.S. Pat. No. 6,928,325, the content of which is herein incorporated by reference. The intravascular flow sensor 350 generates an electrical and/or optical signal corresponding to the velocity of blood flow within a vascular lumen and transmits that signal onto the CRM device 352 through an electric conductor and/or an optical conductor. The signal can be conveyed to a processor 354 wherein the velocity of blood flow is calculated and/or analyzed.

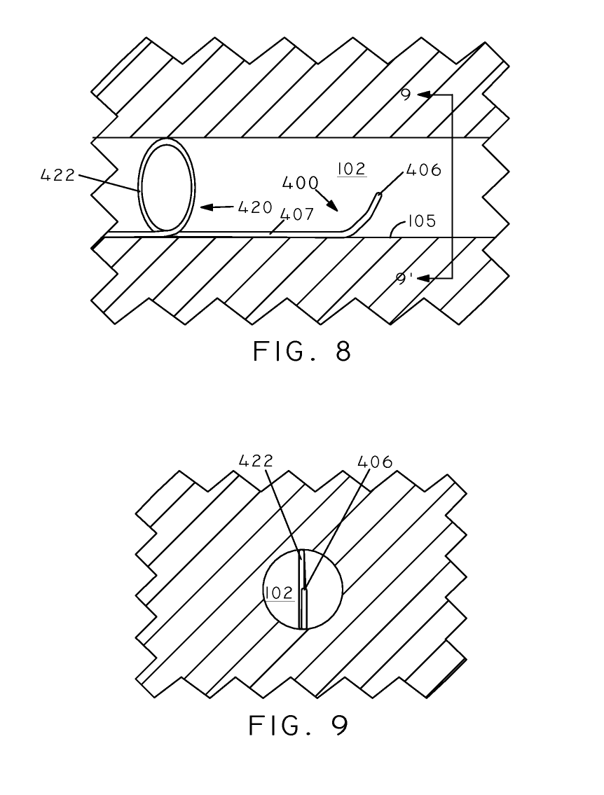

In some embodiments, the intravascular flow sensor is not directly tethered to a CRM device. FIG. 7 shows a schematic diagram of a intravascular flow sensor 370 in conjunction with a CRM device 372, wherein the flow sensor 370 is not directly tethered to the CRM device 372. The intravascular flow sensor 370 can generate a signal that is then transmitted wirelessly between a first transmitter 380 and a second transmitter 378. The signal can be conveyed to a wireless communication module 376 (which can be integral with the second transmitter 378) and then on to a processor 374 for further analysis.

It will be appreciated that the velocity of fluid flow through a conduit with a roughly circular cross-section is not uniform. Specifically, because of drag forces exerted by the conduit walls on the fluid, the average velocity near the conduit walls will generally be less than the average velocity in the center of the conduit in cross-section. In addition, it will be appreciated that the accuracy of a flow sensor may be compromised if the tip of the sensor is disposed against the wall of the vascular lumen. Specifically, if the tip is contacting a stationary element, such as the vascular wall, the tip may be physically constrained and thus may not be able to exhibit flexion that is proportional to the velocity of the blood flow. In some embodiments, the flow sensor of the invention includes a positioning element. In some aspects, the positioning element can function to position the flow sensor within the vascular lumen. In some embodiments, the positioning element can also function to prevent portions of the flow sensor, such as a tip, from contacting the tissue wall surrounding the vascular lumen.

In some embodiments, the positioning element can be configured to exhibit a relatively constant force (or strain tension) outward against the tissue wall surrounding the vascular lumen. As such, even where the inner diameter of the vascular lumen increased in size, acutely or chronically, the positioning element would continue to engage the wall of the vascular lumen. In some embodiments, the positioning element can be configured to exhibit a force outward against the tissue wall that is sufficient to enlarge the cross-sectional area of the lumen in which it is disposed. In some embodiments, the positioning element can enlarge the cross-sectional area in an amount equal to or greater than the cross-sectional area taken up by the intravascular flow sensor itself such that impedance on fluid flow through the vascular lumen caused by the intravascular flow sensor is reduced or minimized.

Referring now to FIG. 8, a schematic side view of a flow sensor 400 with a positioning element 420 is shown within a vascular lumen 102. The vascular lumen 102 is surrounded by a continuous vascular lumen wall 105. A positioning element 420 including a looped structure 422 is attached to the connecting shaft 407 of the flow sensor 400. When the looped structure 422 is in contact with the vascular lumen wall 105, it can position the tip 405 of the flow sensor 400 approximately in the center of the vascular lumen 102. In some embodiments, the looped structure 422 can be expandable such that it expands to the size of the intravascular lumen 102 to ensure that it contacts the vascular lumen wall 105. The looped structure 422 can be the same material as the connecting shaft 407 or it can be a different material. The looped structure 422 can include various materials including polymers, metals, and the like. In some embodiments, the looped structure 422 includes stainless steel. In some embodiments, the looped structure 422 includes a shape-memory metal. In some embodiments, the looped structure 422 includes the alloy Nitinol.

The looped structure 422 can take on various configurations in three dimensions. FIG. 9 shows a front view of a flow sensor taken along line 9-9′ of FIG. 8. In this embodiment, the looped structure 422 has a loop that is disposed within a plane passing only through the middle of the intravascular lumen, such that only certain segments of the looped structure 422 contact the wall of the vascular lumen 102. However, in other embodiments, the looped structure can have a corkscrew type shape wherein the entire looped structure contacts the wall 105 of the vascular lumen 102.

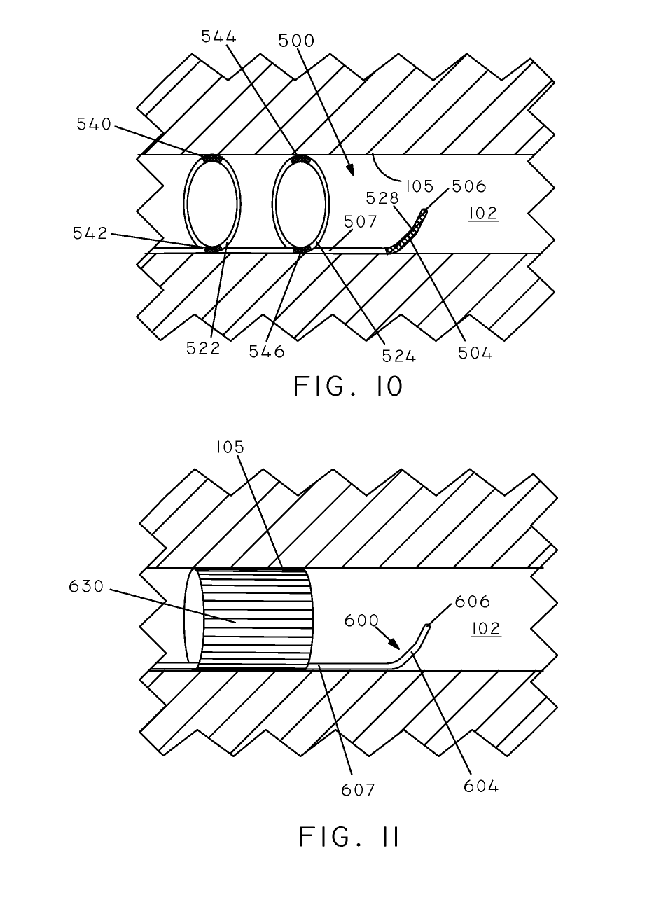

In some embodiments, multiple loop structures can be used to position the tip of the flow sensor. Referring now to FIG. 10, a side view of a flow sensor 500 is shown that includes a first positioning loop 522 and a second positioning loop 524. The first positioning loop 522 and the second positioning loop 524 are attached to the flow sensor connecting shaft 507 in a manner so as to keep the tip 506 of the flow sensor positioned roughly in the middle of the intravascular lumen 102. In some embodiments, the flow sensor 500 can be selectively coated with materials that either aid in tissue attachment or inhibit tissue growth. By way of example, the portions 526 of the first and second positioning loops that contact the wall 105 of the vascular lumen can be coated with a material that aids in tissue attachment, such as a growth factor or other growth promoting substance. Over time, tissue growth can help to secure the positioning loops 522, 524 to the wall of the vascular lumen 102, and therefore secure the flow sensor 500 in place. In some embodiments, a coating 528 can be provided over the tip 506 and the flexible shaft 504 of the flow sensor 500 that prevents the growth of tissue. By way of example, the coating 528 can be configured to elute an agent that inhibits tissue growth. In this manner, tissue growth around the tip of the flow sensor that would otherwise act to decrease the sensitivity of the sensor can be minimized.

It will be appreciated that embodiments of positioning elements can take on various shapes and forms. Referring now to FIG. 11, a cylindrical positioning element 630 is shown in conjunction with a flow sensor 600. The cylindrical positioning element 630 can be attached to the connecting shaft 607 of the flow sensor 600. The cylindrical positioning element 630 can include a solid cylindrical wall or a cylindrical wall defining a plurality of apertures, such as a mesh-type wall. In some embodiments, the cylindrical positioning element 630 can include a stent. In the embodiment shown in FIG. 11, the flexible shaft 604 and the tip 606 of the flow sensor 600 are positioned outside of the cylindrical positioning element 630. However, in some embodiments, the flexible shaft 604 and/or the tip 606 can be positioned within the cylindrical positioning element 630.

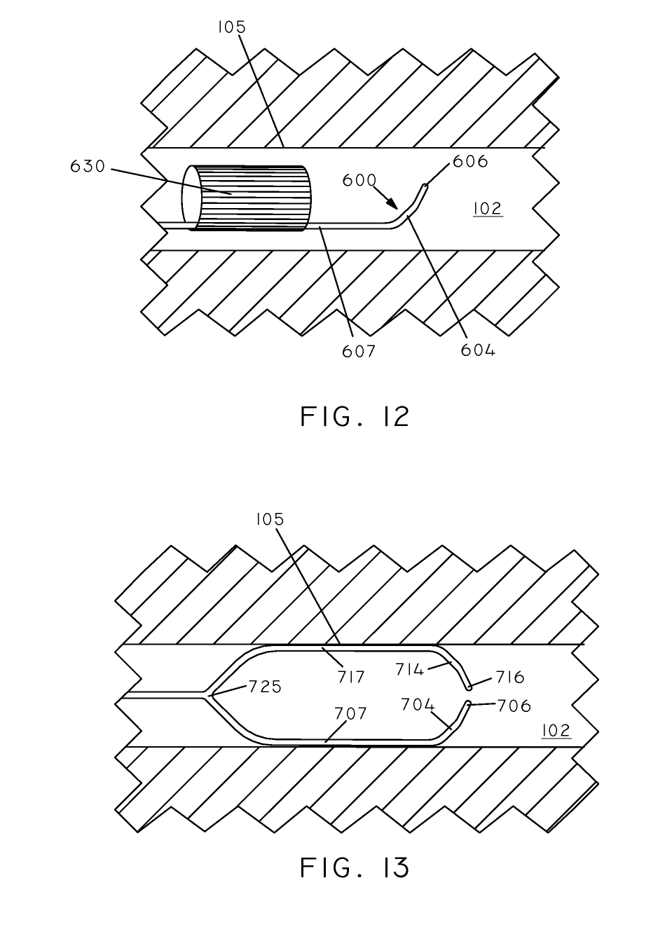

The cylindrical positioning element 630 can be made of various materials including polymers, metals, and the like. In some embodiments, the cylindrical positioning element includes stainless steel. In some embodiments, the cylindrical positioning element 630 includes a shape-memory metal. In some embodiments, the cylindrical positioning element 630 includes a nickel-titanium alloy such as Nitinol. The cylindrical positioning element can be configured to be expandable so as to be inserted into the vasculature using percutaneous surgical techniques and then later expanded so that the walls of the positioning element engage the wall of the vascular lumen and hold the flow sensor 600 in place. FIG. 11 shows the cylindrical positioning element 630 in an expanded configuration with the cylindrical positioning element having a relatively larger diameter so that it engages the walls 105 of the vascular lumen 102. FIG. 12 shows the cylindrical positioning element 630 in an unexpanded configuration with the cylindrical positioning element having a relatively smaller diameter so that the device can be inserted by passing through the vasculature until it is in a desired position. In some embodiments, the cylindrical positioning element is self-expanding. In other embodiments, the cylindrical positioning element is configured to be expanded by a balloon.

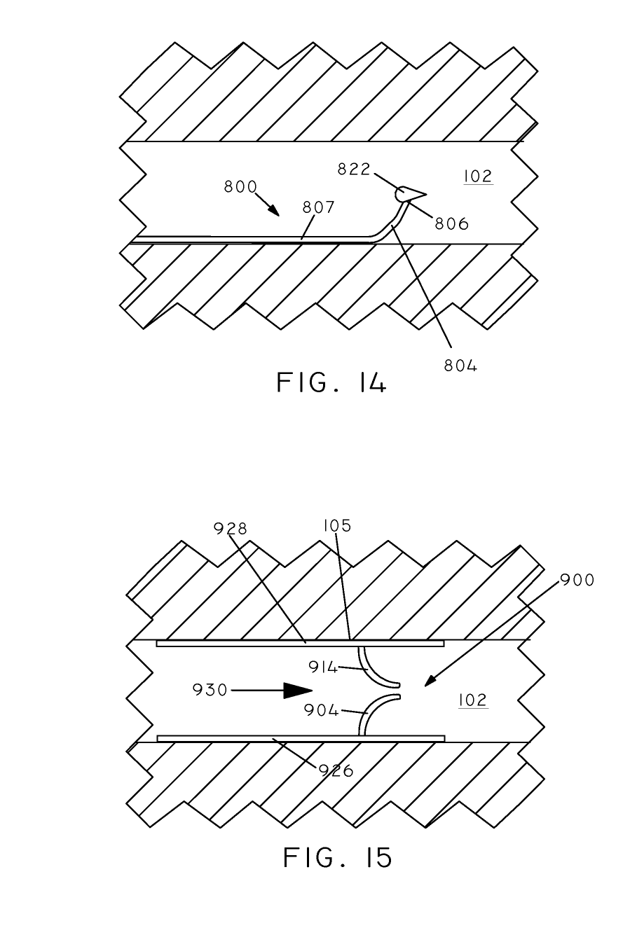

In some embodiments, the flow sensor can include a plurality of flexible shafts and tips. Referring now to FIG. 13, a flow sensor 700 is shown than includes a first connecting shaft 707 connected to a first flexible shaft 704 and a first tip 706. The flow sensor 700 also includes a second connecting shaft 717 connected to a second flexible shaft 714 and a second tip 716. The first connecting shaft 707 and the second connecting shaft 717 are connected at a connection point 725. The connection point 725 and connecting shafts 707, 717 can be configured so that the connecting shafts 707, 717 exert a force outward (away from one another) on opposite sides of the wall 105 of the vascular lumen 102. In this manner, the connecting shafts 707, 717 can serve to center the tips 706, 716 of the flow sensor within the central area of the vascular lumen 102 and prevent the tips 706, 716 from contacting the wall 105 of the vascular lumen 102. In the embodiment shown in FIG. 13, two connecting shafts are included. However, in other embodiments more connecting shafts, including or not including associated flexible shafts and tips, can be used. In some embodiments, between two and thirty-two connecting shafts can be included. In addition to providing a manner of centering the tips in the vascular lumen 102, such a configuration can provide independent measures of flow velocity (for each flexible shaft included) such that accuracy of the velocity measurement can be increased.

In some embodiments, the tip of the flow sensor can be fitted with a drag modifier for purposes of modifying the flow of fluid (such as blood) around the tip. Referring now to FIG. 14, a schematic side view of a flow sensor 800 is shown within a vascular lumen 102. The flow sensor 800 includes a connecting shaft 807 attached to a flexible shaft 804 and a tip 806. A drag modifier 822 is attached to the tip and has a shape that modifies the flow of fluid around the tip 806. In the embodiment shown, the drag modifier 822 has a tear-drop shape that can function to reduce drag on the tip 806 by effectively reducing the drag coefficient (C_{d}) of the tip 806 and flexible shaft 804. The tear-drop shape can reduce drag by minimizing the formation of eddy currents, amongst other effects. In some cases, the drag modifier 822 can be configured to reduce turbulence around the flow sensor in order to reduce potential thrombogenicity or hemolysis of the flow sensor. In vascular lumens that normally have very low flow velocities, sensitivity of the flow sensor can be increased by increasing the drag coefficient of the tip and flexible shaft. In some embodiments, the drag modifier 822 can have a shape that effectively increases the drag coefficient (C_{d}) of the tip 806 and flexible shaft 804.

In some embodiments, the drag modifier 822 can be shaped so as to promote positioning of the tip 806 within the central area of the vascular lumen 102. For example, the drag modifier 822 can have a shape similar to the cross-section of an airfoil so that lift forces favor positioning of the tip 806 within the center of the vascular lumen.

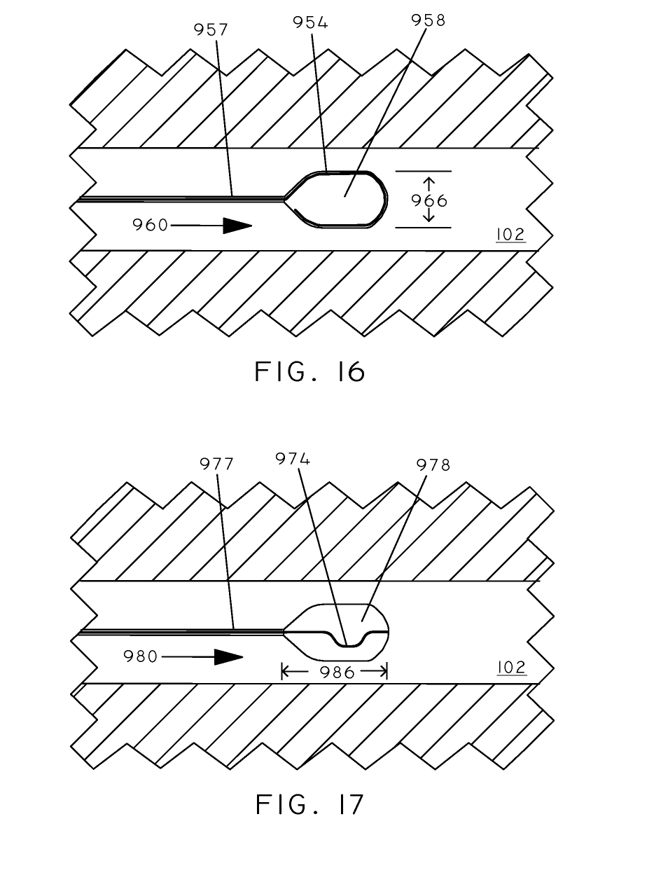

It will be appreciated that components of the flow sensor can take on various shapes and configurations. By way of example, FIG. 15 shows a schematic side view of a flow sensor 900 disposed within a vascular lumen 102 in accordance with another embodiment of the invention. The flow sensor 900 includes a first flexible shaft 904 and a second flexible shaft 914. In this embodiment, the first and second flexible shafts 904, 914 are oriented so as to exhibit flexion in response to blood flowing in the direction of arrow 930. The first flexible shaft 904 is attached to a first wall member 926 and the second flexible shaft 914 is attached to a second wall member 928. The first and second wall members 926 and 928 can be attached to one another or they can be separate. The first and second wall members 926, 928 can be disposed against the wall 105 of the vascular lumen 102 and can act as positioning members, keeping the tips of the flexible shafts 904 and 914 near the center of the vascular lumen 102.

In some embodiments, the flow sensor can include a deformable enclosed volume. The deformable enclosed volume can include, and/or be enclosed by, a deformable member such as a fluid-filled bag, a flexible wall, or a deformable solid such as an elastomeric polymer. By way of example, FIG. 16 shows a flow sensor disposed within a vascular lumen 102 in accordance with another embodiment of the invention including an enclosed volume. The deformable enclosed volume can be of various shapes including ellipsoidal, tear-drop, cylindrical, frusto-conical, and the like. The flow sensor includes a shaft 957 connected to an enclosed volume 958 having a width 966. The enclosed volume 958 can be enclosed by a flexible biocompatible material and can be filled with a biocompatible fluid such as saline. A flexible element 954 is disposed adjacent to the walls of the enclosed volume 958. This flow sensor embodiment can also include one or more positioning elements, such as those described above.

As blood flows through the lumen 102 in the direction of arrow 960, the enclosed volume 958 can deform. Specifically, the width 966 of the enclosed volume 958 can become smaller in response to higher velocities of blood flow in the direction of arrow 960 and this can cause the flexible element 954 to flex. The enclosed volume 958 can exhibit a degree of elasticity such that when the velocity of blood flow diminishes, the width 966 of the enclosed volume increases. The elasticity can be provided in various ways such as by the wall of the enclosed volume, a fluid within the enclosed volume, the flexible element, or another component configured to provide elasticity. The flexible element 954 can include components such as an optical or electrical strain gauge that can generate a signal corresponding to flexion of flexible element 954. This signal can then be evaluated in order to calculate fluid flow velocity through the lumen 102.

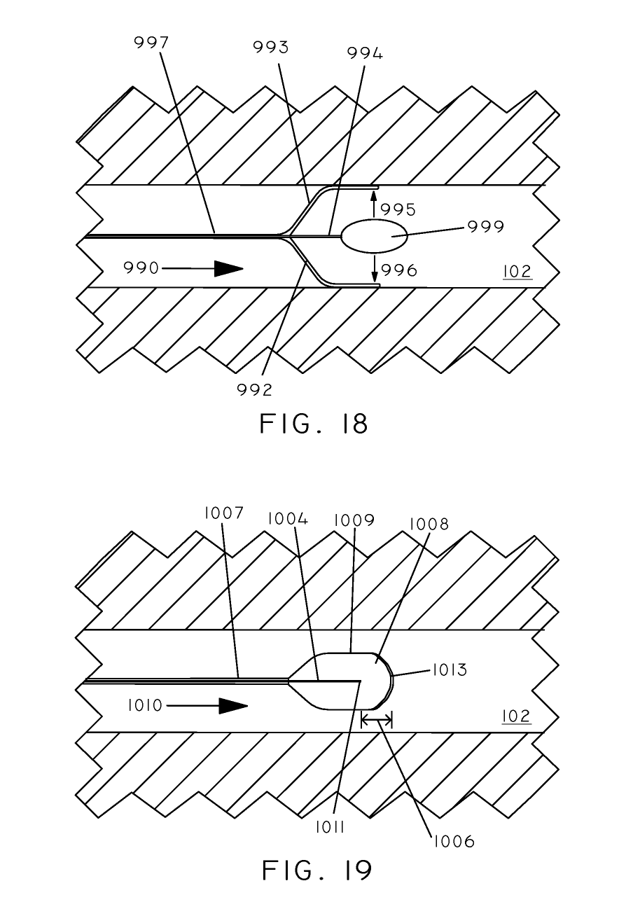

FIG. 17 shows a flow sensor disposed within a vascular lumen 102 in accordance with another embodiment of the invention. The flow sensor includes a shaft 977 connected to an enclosed volume 978 having a length 986. The enclosed volume 978 can be enclosed by a flexible biocompatible material and can be filled with a biocompatible fluid such as saline. A flexible element 974 is disposed within the enclosed volume 978. As blood flows through the lumen 102 in the direction of arrow 980, the enclosed volume 978 can deform. Specifically, the length 986 of the enclosed volume 978 can become longer in response to higher velocities of blood flow in the direction of arrow 980 and this can cause the flexible element 974 to flex. The flexible element 974 can include components such as an optical or electrical strain gauge that can generate a signal corresponding to flexion of flexible element 974. This signal can then be evaluated in order to calculate fluid flow velocity through the lumen 102. This flow sensor embodiment can also include one or more positioning elements, such as those described above.

FIG. 18 shows a flow sensor disposed within a vascular lumen 102 in accordance with another embodiment of the invention. The flow sensor includes a shaft 997 connected to a first positioning member 992 and a second positioning member 993. The flow sensor also includes a flexible element 994 attached to a blocking element 999. As blood flows through the lumen 102 in the direction of arrow 990, the blood encounters blocking element 999 and causes it to be deflected in the direction of arrow 995 or arrow 996 (depending on the shape of blocking element 999) causing flexible element 994 to flex. The flexible element 994 can include components such as an optical or electrical strain gauge that can generate a signal corresponding to flexion of flexible element 994. This signal can then be evaluated in order to calculate fluid flow velocity through the lumen 102.

FIG. 19 shows a flow sensor disposed within a vascular lumen 102 in accordance with another embodiment of the invention. The flow sensor includes a shaft 1007 connected to an enclosed volume 1008. An optical conductor 1004 with a tip 1011 is disposed within the enclosed volume 1008. The flow sensor can include a deformable member such as a wall 1009. The wall 1009 can have a reflective region 1013. The tip 1011 is separated from the reflective region 1013 of the enclosed volume 1008 by a distance 1006. The remainder of the wall 1009 can be optically absorptive. Light can be passed through the optical conductor 1004 and out of the tip 1011 towards the reflective region 1013 of the enclosed volume 1008. The light can then be reflected off the reflective region 1013 and back to the tip 1011 and through the optical conductor 1001. A photo-diode, or a similar device, can be in optical communication with the optical conductor 1001 in order to convert this optical signal into an electrical signal. The enclosed volume 1008 can be filled with a fluid or flexible solid having optical properties such that the greater the distance traveled by light through the enclosed volume 1008, the less light that is returned from the reflective region 1013 and back to the tip 1011. For example, the fluid or flexible solid can cause absorptive or diffusional losses of light. Thus, the amount of light returning to the tip 1011 can depend on the distance 1006 between the tip 1011 and the reflective region 1013. As blood flows through the lumen 102 in the direction of arrow 1010, the enclosed volume 1008 can deform. Specifically, the distance 1006 between the tip 1011 and the reflective region 1013 can increase in response to higher velocities of blood flow in the direction of arrow 1010. In this manner, the velocity of blood flow can be correlated with the amount of light returning to the tip 1011. It will be appreciated that this flow sensor embodiment can also include one or more positioning elements, such as those described above.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as “arranged”, “arranged and configured”, “constructed and arranged”, “constructed”, “manufactured and arranged”, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

This application is intended to cover adaptations or variations of the present subject matter. It is to be understood that the above description is intended to be illustrative, and not restrictive. The scope of the present subject matter should be determined with reference to the appended claims, along with the full scope of equivalents to which such claims are entitled.

What is claimed is:

1. An intravascular flow sensor comprising:
a strain gauge; and
a positioning element operably connected to the strain gauge, the positioning element configured to be expandable from a first diameter to a second diameter, the second diameter larger than the first diameter.

2. The intravascular flow sensor of claim 1, the strain gauge comprising an optical strain gauge.

3. The intravascular flow sensor of claim 1, the strain gauge comprising an electrical strain gauge.

4. The intravascular flow sensor of claim 1, the strain gauge coated with an agent to inhibit the growth of tissue thereon.

5. The intravascular flow sensor of claim 1, the positioning element comprising a cylinder, the cylinder comprising a mesh.

6. The intravascular flow sensor of claim 1, the positioning element comprising a stent.

7. The intravascular flow sensor of claim 1, the positioning element comprising a loop of material.

8. The intravascular flow sensor of claim 7, the loop configured to expand to the diameter of a vascular lumen in which the loop is disposed.

9. The intravascular flow sensor of claim 7, at least two points of the loop configured to contact a wall of a vascular lumen.

10. The intravascular flow sensor of claim 7, a portion of the loop coated with an agent to enhance the growth of tissue.

11. An intravascular flow sensor comprising:
a deflection member configured to be positioned within a lumen defined by a tissue wall, the deflection member comprising a flexible shaft and a shaft tip, the deflection member configured to generate a signal corresponding to flexion of the flexible shaft; and
a positioning member configured to engage the tissue wall and prevent the shaft tip from contacting the tissue wall.

12. The intravascular flow sensor of claim 11, the positioning member comprising a loop of material.

13. The intravascular flow sensor of claim 11, the positioning member comprising a cylinder.

14. The intravascular flow sensor of claim 11, the positioning member configured to expand in diameter to engage the tissue wall.

15. The intravascular flow sensor of claim 11, further comprising a drag-modifier attached to the shaft tip, the drag-modifier configured to reduce the generation of eddy-currents by the flow sensor.

16. The intravascular flow sensor of claim 11, the intravascular flow sensor further comprising a conductor for transferring the signal from the deflection member to an implantable cardiac rhythm management device.

17. The intravascular flow sensor of claim 11, the signal comprising an optical signal.

18. The intravascular flow sensor of claim 11, the signal comprising an electrical signal.

19. An implantable medical device comprising:
a pulse generator; and
an intravascular flow sensor in communication with the pulse generator, the intravascular flow sensor comprising a strain gauge.

20. The implantable medical device of claim 19, the intravascular flow sensor defining an enclosed volume.

21. An intravascular flow sensor comprising:
a deformable member enclosing a volume; and
a sensor disposed within the enclosed volume, wherein deformation of the enclosed volume results in the generation of a signal.

22. The intravascular flow sensor of claim 21, the enclosed volume filled with a fluid.

23. The intravascular flow sensor of claim 21, the sensor comprising an optical sensor, the signal comprising an optical signal.

24. The intravascular flow sensor of claim 23, the optical sensor comprising an optical strain gauge.

25. The intravascular flow sensor of claim 23, the optical sensor comprising an optical fiber, the optical fiber comprising a tip, the deformable member comprising a wall, the tip separated from the wall by a gap.

26. The intravascular flow sensor of claim 25, configured so that deformation of the deformable member in response to increased flow velocity increases the size of the gap between the tip and the wall.
